(19) United States
(12) Patent Application Publication
(10) Pub. No.: US 2020/0345795 A1
(43) Pub. Date: Nov. 5, 2020

(54) MODULATION OF INTESTINAL MICROBIOTA IN PRE-DIABETES AND TYPE 2 DIABETES

(71) Applicant: Medlab IP Pty Ltd, New South Wales (AU)

(72) Inventors: Luis VITETTA, Queensland (AU); Talia PALACIOS, New South Wales (AU)

(73) Assignee: Medlab IP Pty Ltd, New South Wales (AU)

(21) Appl. No.: 16/760,577

(22) PCT Filed: Nov. 1, 2018
(86) PCT No.: PCT/AU2018/051183

§ 371 Date: Apr. 30, 2020

(30) Foreign Application Priority Data
Nov. 1, 2017 (AU) 2017904443

(51) Int. Cl. A61K 35/744; A61K 31/155; A61P 3/10; A61K 35/747; A61K 35/745; A61K 36/064

(52) U.S. Cl. CPC A61K 35/744; A61K 31/155; A61P 3/10; A61K 2035/115; A61K 35/745; A61K 36/064; A61K 35/747

(57) ABSTRACT

Provided herein are methods for modulating the intestinal microbial profile and for reducing intestinal dysbiosis in type 2 diabetic or pre-diabetic subjects, comprising administering metformin or a derivative or salt thereof and one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii. Also provided are methods for treating type 2 diabetes or an associated metabolic complication or condition and for delaying the onset of type 2 diabetes in pre-diabetic subjects, comprising administering one or more probiotic microorganisms selected from Lactobacillus plantarum, Lactobacillus bulgaricus, Lactobacillus gasseri, Bifidobacterum breve, Bifidobacterium animalis sub sp. lactis, Bifidobacterium bifidum, Streptococcus thermophilus and Saccharomyces boulardii.

### FIELD OF THE ART

The present disclosure relates generally to methods for modulating the intestinal microbial profile and reducing intestinal dysbiosis in type 2 diabetic subjects or pre-diabetic subjects using metformin and a multi-strain probiotic combination. The disclosure also relates to methods for treating type 2 diabetes and metabolic complications and conditions associated therewith, and for delaying or inhibiting the onset of type 2 diabetes.

### BACKGROUND

Type 2 diabetes mellitus is a chronic metabolic disorder characterised by impaired β-cell function, insulin resistance and typically an increasing inability to synthesise sufficient insulin. Type 2 diabetes is a common disease of increasing prevalence worldwide, strongly associated with obesity. Type 2 diabetes is also often associated with macrovascular complications such as cardiovascular disease, and/or microvascular complications such as blindness, neuropathy and/or renal impairment or failure.

Treatment of type 2 diabetes can include the administration of agents that stimulate β-cell function or that enhance the tissue sensitivity to insulin. Agents known to stimulate β-cell function, include, for example, sulfonylureas, such as tolbutamide, glibenclamide, glipizide, chlorpropamide, and gliclazide, and repaglinide. Metformin (N,N-dimethylimidodicarbonimidic diamide or 1,1-dimethylbiguanide) is an antihyperglycemic agent which improves glucose tolerance in patients with type 2 diabetes. It is the most widely used medication to improve glycemic control in type 2 diabetics, and has been shown to reduce body weight and delay the onset of type 2 diabetes in at risk or pre-diabetic individuals.

However existing treatments for type 2 diabetes can often lead to unsatisfactory results for individuals and may be associated with adverse side effects. There is a clear and growing need for the development of alternative or adjunct treatments of type 2 diabetes with the increasing prevalence of the disease.

The relationship between the gastrointestinal microbiota and obesity-associated disorders has gained increasing interest as our understanding of the importance of the gastrointestinal microbiome has grown. A disturbed intestinal microbiota (intestinal dysbiosis) has been associated with the progression and maintenance of obesity, type 2 diabetes mellitus, cardiovascular disease and metabolic syndrome. While our understanding of the pathogenic effects and associations of intestinal dysbiosis has grown, there is a clear need for the development of methods and therapies to address this dysbiosis, improve the intestinal microbiota and thus assist in the treatment of a variety of diseases.

### SUMMARY OF THE DISCLOSURE

In a first aspect, the present disclosure provides a method for modulating the intestinal microbial profile in a type 2 diabetic or pre-diabetic subject, the method comprising administering to the subject metformin or a derivative or salt thereof and one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii, whereby the modulation of the intestinal microbial profile resulting from said administration comprises a decrease in abundance of pathogenic bacteria or bacteria that do not tolerate the host and/or an increase in the abundance of bacteria that tolerate the host.

The pathogenic bacteria may be opportunistic pathogens. In an embodiment, the bacteria that do not tolerate the host are pathobiont bacteria. In an embodiment, the bacteria that tolerate the host are beneficial bacteria normally resident in the gastrointestinal tract, such as those capable of producing or increasing local production of short chain fatty acids.

In an embodiment, the metformin or derivative or salt thereof is administered in conjunction with a multi-strain probiotic combination comprising Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

The metformin or derivative or salt thereof and the one or more probiotic microorganisms or the multi-strain probiotic combination may be administered simultaneously or sequentially. For simultaneous administration the metformin or derivative or salt thereof and the one or more probiotic microorganisms or the multi-strain probiotic combination may be in the same or different compositions.

Either or both of the metformin or derivative or salt thereof and the one or more probiotic microorganisms or the multi-strain probiotic combination may be present in a capsule. In exemplary embodiments the one or more probiotic microorganisms or the multi-strain probiotic combination are administered at a dose of about 5×10⁵ cfu, once or twice daily.

In an embodiment the intestinal microbial profile is determined from one or more faecal samples from the subject. Said determination may comprise rRNA gene sequencing.

Modulation of the intestinal microbial profile may comprise an increase in abundance of Bifidobacterium spp., Phascoarctobacterium spp. and/or Akkermansia municiphila. Modulation of the intestinal microbial profile may comprise a decrease in abundance of Roseburia spp. such as Roseburia intestinalis and/or Roseburia inulinivorans.

In a second aspect, the present disclosure provides a method for reducing intestinal dysbiosis in a type 2 diabetic or pre-diabetic subject, the method comprising administering to the subject metformin or a derivative or salt thereof and one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

In an embodiment, the metformin or derivative or salt thereof is administered in conjunction with a multi-strain probiotic combination comprising Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

In a third aspect, the present disclosure provides a method for treating type 2 diabetes, or a metabolic complication or condition associated therewith, the method comprising administering to a subject one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

In an embodiment, the subject is administered a multi-strain probiotic combination comprising Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

Optionally the subject is also administered metformin or a derivative or salt thereof.

Treating the type 2 diabetes or metabolic complication or condition associated therewith may comprise decreasing glycosylated haemoglobin (HbA1c) levels, reducing BMI, improving insulin sensitivity, for example as determined by the Matsuda Index of Insulin Sensitivity (ISI-M), or reducing insulin resistance, for example as determined by the Homeostatic Model Assessment of Insulin Resistance (HOMA-IR).

In a fourth aspect, the present disclosure provides a method for delaying or inhibiting the onset of type 2 diabetes in a pre-diabetic subject, the method comprising administering to the subject one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

In an embodiment, the subject is administered a multi-strain probiotic combination comprising Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

Optionally the subject is also administered metformin or a derivative or salt thereof.

Delaying or inhibiting the onset of type 2 diabetes may comprise decreasing glycosylated haemoglobin (HbA1c) levels, reducing BMI, improving insulin sensitivity, for example as determined by the Matsuda Index of Insulin Sensitivity (ISI-M), or reducing insulin resistance, for example as determined by the Homeostatic Model Assessment of Insulin Resistance (HOMA-IR).

The above methods may further comprise administering to the subject one or more prebiotic components. In an exemplary embodiment the prebiotic is FOS or a resistant starch. The prebiotic may be present in the same composition or formulation as one or more of the probiotic strains or may be present in a different composition or formulation.

In accordance with the above aspects, the metformin or derivative or salt thereof and the one or more probiotic microorganisms or multi-strain probiotic combination may be present in a single composition.

Accordingly, also provided herein is a composition for treating type 2 diabetes, for delaying or inhibiting the onset of type 2 diabetes, or for modulating the intestinal microbiota in a type 2 diabetic or pre-diabetic subject, the composition comprising metformin or a derivative or salt thereof and one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

### BRIEF DESCRIPTION OF THE DRAWINGS

Embodiments of the disclosure are described herein, by way of non-limiting example only, with reference to the accompanying drawings.

FIG. 1 Principal component analysis plots of the effect of metformin on intestinal microbiota in study participants. Analysis performed in all the participants (prediabetes+T2DM) and in the subgroups (prediabetic and T2DM) at the species (A), genus (B) and family (C) levels.

FIG. 2 Principal component analysis plots of the effect of the multi-strain probiotic combination on intestinal microbiota in study participants taking metformin at the species (A), genus (B) and family (C) levels.

### DETAILED DESCRIPTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

In the context of this specification, the term “probiotic” is to be given its broadest construction and is understood to refer to a microbial cell population or preparation, or component of a microbial cell population or preparation, which when administered to a subject in an effective amount promotes a health benefit in the subject.

In the context of this specification, the term “prebiotic” is to be given its broadest construction and is understood to refer to any non-digestible substance that stimulates the growth and/or activity of bacteria in the digestive system.

In the context of this specification, the terms “food”, “foods”, “beverage” or “beverages” include but are not limited to health foods and beverages, functional foods and beverages, and foods and beverages for specified health use. When such foods or beverages of the present invention are used for subjects other than humans, the terms can be used to include a feedstuff.

The term “subject” as used herein refers to any mammal, including, but not limited to, livestock and other farm animals (such as cattle, goats, sheep, horses, pigs and chickens), performance animals (such as racehorses), companion animals (such as cats and dogs), laboratory test animals and humans. Typically the subject is a human.

As used herein, the term “effective amount” refers to an amount of metformin or a derivative or salt thereof, or of one or more probiotic microorganisms or a multi-strain probiotic combination that is sufficient to effect one or more beneficial or desired outcomes. An “effective amount” can be provided in one or more administrations. The exact amount required will vary depending on factors such as the form of metformin administered, the identity and number of individual probiotic strains employed, the subject being treated, the nature of the disease(s) or condition(s) suffered by the subject that is to be treated and the age and general health of the subject, and the form in which the composition is administered. Thus, it is not possible to specify an exact “effective amount”. However, for any given case, an appropriate “effective amount” may be determined by one of ordinary skill in the art using only routine experimentation.

As used herein the terms “treating”, “treatment” and the like refer to any and all applications which remedy, or otherwise hinder, retard, or reverse the progression of, a disease or disorder or at least one symptom of a disease or disorder, including reducing the severity of a disease or disorder. Thus, treatment does not necessarily imply that a subject is treated until complete recovery from a disease or disorder. Similarly, the terms “preventing”, “prevention” and the like refer to any and all applications that prevent the establishment of a disease or disorder or otherwise delay the onset of a disease or disorder.

As used herein the terms “reducing”, “reduce” and the like in the context of intestinal dysbiosis refers to a normalization of the intestinal microbiota or the intestinal microbial profile in a subject, wherein normalization means restoring (e.g. the abundance) of the intestinal microorganisms to or towards that expected, or observed in individuals, in the absence of pre-diabetes, type 2 diabetes, insulin resistance or reduced insulin-sensitivity.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

Provided herein are methods for modulating the intestinal microbial profile in type 2 diabetic or pre-diabetic subjects, comprising administering metformin or a derivative or salt thereof and one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii, whereby the modulation of the intestinal microbial profile resulting from said administration comprises a decrease in abundance of pathogenic bacteria or bacteria that do not tolerate the host and/or an increase in the abundance of bacteria that tolerate the host.

Also provided are methods for reducing intestinal dysbiosis in type 2 diabetic or pre-diabetic subjects, comprising administering metformin or a derivative or salt thereof and one or more probiotic microorganisms selected from Lactobacillus plantarum, Lactobacillus bulgaricus, Lactobacillus gasseri, Bifidobacterum breve, Bifidobacterium animalis sub sp. lactis, Bifidobacterium bifidum, Streptococcus thermophilus and Saccharomyces boulardii.

Without wishing to be bound by theory, the inventors suggest that modulation of the intestinal microbial profile or reduction of intestinal dysbiosis (i.e. normalization of the intestinal microbiota towards that observed in individuals without pre-diabetes or type 2 diabetes) in accordance with the present disclosure to restore homeostasis may be beneficial in treating or controlling type 2 diabetes and associated metabolic disorders. In exemplary embodiments, modulating the intestinal microbial profile may comprise an increase in abundance of beneficial intestinal microorganisms such as bacteria that produce, or can increase the local production or levels of short chain fatty acids such as acetic acid, propionic acid or butyric acid. In exemplary embodiments, modulating the intestinal microbial profile may comprise an increase in abundance of beneficial intestinal microorganisms including Bifidobacterium spp., Phascoarctobacterium spp. and Akkermansia spp. such as Akkermansia municiphila. Modulation of the intestinal microbial profile may comprise a decrease in abundance of pathogenic microorganisms including opportunistic pathogens such as E. coli and Egerthella lenta, and of Roseburia spp. such as Roseburia intestinalis and/or Roseburia inulinivorans. Modulation of the intestinal microbial profile may comprise a decrease in abundance of pathobiont bacteria.

Metformin can exist as a free base, or may form salts, including pharmaceutically acceptable salts, such as a hydrochloride salt (e.g., a mono-hydrochloride salt. Accordingly, in accordance with the present disclosure, the metformin may be administered as the free base form, or as a hydrochloride salt, a mono-hydrochloride salt or other a pharmaceutically acceptable salt of metformin. Suitable metformin salts include, but are not limited to, metformin hydrochloride, metformin glycinate salts, metformin fumarate salts and metformin succinate salts. Other representative pharmaceutically acceptable salts include, for example, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, gluceptate, gluconate, glutamate, glycollylarsanilate, hexylresorcinate, hydrabamine, hydrobromide, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, tannate, tartrate, teoclate, tosylate, triethiodide, trimethylammonium and valerate. The skilled addressee will appreciate that the scope of the present disclosure is not limited by reference to specific salts of metformin.

Also contemplated herein are derivatives of metformin, including synthetic derivatives of metformin such as HL010183 and other derivatives such as those described in U.S. Pat. No. 8,853,259 (the disclosure of which is incorporated herein by reference). The skilled addressee will appreciate that the scope of the present disclosure is not limited by reference to specific derivatives of metformin.

The metformin or derivative or salt thereof may be included in any suitable dosage form. For example, the metformin may exist in a powder, a tablet, a capsule, or the like. Such dosage forms may, in some embodiments, also include specialized coatings, matrices, and the like to give effect a sustained release, a controlled release, enteric release, etc. In some embodiments, the metformin or derivative or salt thereof may be present in a dosage form with the one or more probiotic microorganisms or multi-strain probiotic combination.

The metformin or derivative or salt thereof may be administered in any dosage suitable for the treatment of type 2 diabetes. Such dosages will be well known to the skilled person. For example, in an immediate or controlled release formulation, the metformin or derivative or salt thereof may be administered in an initial dose of between about 500 mg to 1000 mg daily, increasing to a maintenance dose of about 2000 mg daily. The daily amount may be administered as a single dose or in divided doses two or three times a day. By way of example only, the daily dose of metformin or derivative or salt thereof administered in accordance with the present disclosure may be between about 500 mg and about 2500 mg, such as about 500 mg, about 700 mg, about 900 mg, about 1100 mg, about 1300 mg, about 1500 mg, about 1700 mg, about 1900 mg, about 2100 mg, about 2300 mg or about 2500 mg.

In particular embodiments, the probiotic microorganisms are administered as a multi-strain probiotic combination comprising Lactobacillus plantarum, Lactobacillus bulgaricus, Lactobacillus gasseri, Bifidobacterum breve, Bifidobacterium animalis sub sp. lactis, Bifidobacterium bifidum, Streptococcus thermophilus and Saccharomyces boulardii. Optionally the one or more probiotic microorganisms or the multi-strain combination may be present in a composition as specially selected strains as a culture concentrate or as part of a multi-strain blend, optionally with a variety of excipients.

Methods of the present disclosure may further comprise the administration of one or more additional probiotic microorganisms selected from, for example, Lactobacillus reuteri, Lactobacillus paracasei, Lactobacillus casei, Lactobacillus fermentum, Lactobacillus salvarius, Lactobacillus delbrueckii spp. bulgaricus, Lactobacillus helveticus, Lactobacillus johnsonii, Lactococcus lactis, Bifidobacterium animalis subsp. animalis (B. animalis), Bifidobacterium infantis, Bifidobacterium longum, Bifidobacterium adolescentis and Bifidobacterium pseudocatenulatum.

The amounts of individual microorganisms to be administered to subjects or to be included in compositions disclosed herein will depend on a variety of factors including the identity and number of individual strains employed, the condition or disease to be treated or against which the composition is designed to be used, and the form in which a composition is administered. For any given case, appropriate amounts may be determined by one of ordinary skill in the art using only routine experimentation. By way of example only, the amount of each microbial strain present in a single dose of a composition disclosed herein may be from about 1×10² cfu to about 1×10¹¹ cfu, and may be about 1×10³ cfu, about 2.5×10³ cfu, about 5×10³ cfu, about 7.5×10³ cfu, 1×10⁴ cfu, about 2.5×10⁴ cfu, about 5×10⁴ cfu, about 7.5×10⁴ cfu, about 1×10⁵ cfu, about 2.5×10⁵ cfu, about 5×10⁵ cfu, about 7.5×10⁵ cfu, about 1×10⁶ cfu, about 2.5×10⁶ cfu, about 5×10⁶ cfu, about 7.5×10⁶ cfu, about 1×10⁷ cfu, about 2.5×10⁷ cfu, about 5×10⁷ cfu, about 7.5×10⁷ cfu, about 1×10⁸ cfu, about 2.5×10⁸ cfu, about 5×10⁸ cfu, about 7.5×10⁸ cfu, about 1×10⁹ cfu, about 2.5×10⁹ cfu, about 5×10⁹ cfu, about 7.5×10⁹ cfu, about 1×10¹⁰ cfu, about 2.5×10¹⁰ cfu, about 5×10¹⁰ cfu, about 7.5×10¹⁰ cfu, and about 1×10¹¹ cfu.

In embodiments in which the metformin or derivative or salt thereof is formulated in a single composition with the one or more probiotic microorganisms, it is well within the skills and capabilities of the skilled person to determine, without undue burden, the amounts of each component to be incorporated into the composition, for example into a unit dosage form, to enable the administration of the appropriate daily dose of each component as described herein.

Also contemplated by the present disclosure are variants of the microorganisms described herein. As used herein, the term “variant” refers to both naturally occurring and specifically developed variants or mutants of the microbial strains disclosed and exemplified herein. Variants may or may not have the same identifying biological characteristics of the specific strains exemplified herein, provided they share similar advantageous properties in terms of their ability to be used as probiotic strains. Illustrative examples of suitable methods for preparing variants of the microbial strains exemplified herein include, but are not limited to, culturing under selective growth conditions, gene integration techniques such as those mediated by insertional elements or transposons or by homologous recombination, other recombinant DNA techniques for modifying, inserting, deleting, activating or silencing genes, intraspecific protoplast fusion, mutagenesis by irradiation with ultraviolet light or X-rays, or by treatment with a chemical mutagen such as nitrosoguanidine, methylmethane sulfonate, nitrogen mustard and the like, and bacteriophage-mediated transduction. Suitable and applicable methods are well known in the art and are described, for example, in J. H. Miller, Experiments in Molecular Genetics, Cold Spring Harbor Laboratory Press, Cold Spring Harbor, N.Y. (1972); J. H. Miller, A Short Course in Bacterial Genetics, Cold Spring Harbor Laboratory Press, Cold Spring Harbor, N.Y. (1992); and J. Sambrook, D. Russell, Molecular Cloning: A Laboratory Manual, 3rd ed., Cold Spring Harbor Laboratory Press, Cold Spring Harbor, N.Y. (2001), inter alia.

Also encompassed by the term “variant” as used herein are microorganisms or strains phylogenetically closely related to microorganisms or strains disclosed herein and microorganisms or strains possessing substantial sequence identity with the microorganisms and strains disclosed herein at one or more phylogenetically informative markers such as rRNA genes, elongation and initiation factor genes, RNA polymerase subunit genes, DNA gyrase genes, heat shock protein genes and recA genes. For example, the 16S rRNA genes of a “variant” microorganism or strain as contemplated herein may share about 85%, 86%, 87%, 88%, 89%, 90%, 91%, 92%, 93%, 94%, 95%, 96%, 97%, 98% or 99% sequence identity with a strain disclosed herein.

The microorganisms to be employed in accordance with the present disclosure may be cultured according to any suitable method known to the skilled addressee and may be prepared for addition to a composition by, for example, freeze-drying, spray-drying or lyophilisation. Thus, in embodiments of the present disclosure the microorganisms may be in a dried form (such as lyophilized or sporulated form) in a suitable carrier medium, for example a FOS medium or other soluble fiber, sugar, nutrient or base material for the composition, with which the bacterial strains can be presented in an orally administrable form. One or more of the strains may be encapsulated in, for example, a suitable polymeric matrix to improve long-term stability and storage of the compositions. In one example, encapsulation may comprise alginate beads, although those skilled in the art will appreciate that any suitable encapsulation material or matrix may be used. Encapsulation may be achieved using methods and techniques known to those skilled in the art.

In some embodiments, methods of the present disclosure may comprise administration to the subject of one or more prebiotic components. Similarly, in some embodiments, compositions of the present disclosure may further comprise at least one prebiotic component. Suitable prebiotics include polydextrose, inulin, fructooligosaccharides (FOS), xylooligosaccharides (XOS), galactooligosaccharides (GOS), mannan oligosaccharides, arabinogalacatans (such as larch arabinogalactans), resistant starches (such as Hi-Maize resistant starch), protein-based green lipped mussel extract, and various prebiotic-containing foods such as raw onion, raw leek, raw chickory root and raw artichoke. In certain embodiments the prebiotic component is a fructooligosaccharide. Those skilled in the art will appreciate that other prebiotics may be added to the compositions.

In accordance with particular embodiments of the invention the at least one prebiotic component may be administered or be present in a composition in an amount of from about 1 mg to about 100 g, or more typically between about 5 mg to about 50 g. Alternatively, the composition may comprise about 10 mg, 100 mg, 1 g, 5 g, 10 g, 15 g, 20 g, 25 g, 30 g, 35 g, 40 g or 45 g of prebiotic.

In some embodiments, methods of the present disclosure may comprise administration to the subject of a source of fibre. Similarly, in some embodiments, compositions of the present disclosure may further comprise a source of fibre. The source of fibre may comprise soluble fibre, insoluble fibre, or a combination of both soluble and insoluble fibre. Suitable sources of soluble fibre include, but are not limited to, psyllium, dextrins, fructans, oligosaccharides (e.g. inulins) and polysaccharides. Exemplary polysaccharides include resistant starches and arabinogalactans. An exemplary arabinogalactan is Larch arabinogalactan. The resistant starch may be an RS1, RS2. RS3 or RS4 resistant starch. An exemplary resistant starch is Hi-Maize resistant starch. Arabinogalactans are complex proteoglycans of arabinose and galactose, often classified as plant or microbial arabinogalactan.

Molecules such as acetyl-1-carnitine, alpha-lactalbumin, beta-lactoglobulin, glycomacropeptides, immunoglobulin G, and bovine serum albumin may augment the health of the gut individually and/or in combination with each other as well as with the administration of probiotic bacteria, and optionally a prebiotic, as disclosed herein. This augmented health benefit may translate in a reduction of risk of chronic disease progression via the gastrointestinal control of obesity. Accordingly, embodiments of the present invention contemplate the addition of carnitine, such as acetyl-1-carnitine, and/or a protein-containing component to compositions disclosed herein, and the administration thereof to subjects in accordance with methods of the invention. The protein-containing component may comprise a protein powder, such as a milk powder. The milk powder may be skim milk powder. The protein-containing component may comprise one or more of colostrum or a protein-containing fraction thereof, alpha-lactalbumin, beta-lactoglobulin, glycomacropeptides, lactoferrin, immunoglobulin G and/or bovine serum albumin.

In some embodiments, methods of the present disclosure may comprise administration to the subject of suitable vitamins, minerals and/or amino acids. Similarly, in some embodiments, compositions of the present disclosure may further comprise suitable vitamins, minerals and/or amino acids. The vitamins and minerals may be selected from, but not limited to: vitamins A, B₁, B₂, B₃, B₅, B₆, B₉, B₁₂, C, D, E and calcium, chromium, copper, fluorine, iodine, iron, magnesium, manganese, molybdenum, phosphorus, potassium, selenium, sodium and zinc. The amino acids may be selected from, but are not limited to: alanine, leucine, valine, isoleucine, arginine, aspartic acid, cystine, glycine, histidine, lysine, methionine, phenylalanine, proline, serine, threonine, tryptophan and tyrosine.

In some embodiments, methods of the present disclosure may comprise administration to the subject of one or more antioxidants. Similarly, in some embodiments, compositions of the present disclosure may further comprise one or more antioxidants. The antioxidants may be water-soluble or lipid-soluble antioxidants. Exemplary water soluble antioxidants include sodium ascorbate, calcium ascorbate, potassium ascorbate, ascorbic acid, glutathione, lipoic acid and uric acid. Exemplary lipid soluble antioxidants include tocopherols, tocotrienols, phenols, polyphenols and the like.

Also provided herein are methods for treating type 2 diabetes and metabolic complications and conditions associated therewith, and for delaying or inhibiting the onset of type 2 diabetes, comprising administering to a subject in need one or more probiotic microorganisms selected from, or a multi-strain probiotic combination comprising, Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

Treatments in accordance with the disclosure may comprise improving one or more symptoms or markers of pre-diabetes or type 2 diabetes, or a metabolic complication or condition associated therewith. Such symptoms or markers include, by way of example only, glycosylated haemoglobin (HbA1c) levels, BMI, insulin sensitivity, for example as determined by the Matsuda Index of Insulin Sensitivity (ISI-M), and insulin resistance, for example as determined by the Homeostatic Model Assessment of Insulin Resistance (HOMA-IR). Marker levels may be determined using any suitable method known to those skilled in the art.

Metabolic complications and conditions associated with type 2 diabetes are well known to those skilled in the art, and include for example, diabetic hypoglycaemia, diabetic hyperglycemia, automimmune dysfunction, microvascular conditions such as nephropathy, neuropathy and retinopathy, macrovascular conditions such as peripheral arterial disease, transient ischemic attacks, angina pectoris and myocardial infarction. Also encompassed by metabolic complications and conditions associated with type 2 diabetes as referred to herein include metabolic disorders related to, and associated with, type 2 diabetes such as metabolic syndrome, insulin resistance, hyperglycaemia, hypoglycaemia, hyperinsulinemia and hypoinsulinemia.

Compositions of the disclosure may further comprise any suitable additives, carriers, additional therapeutic agents, bioavailability enhancers, side-effect suppressing components, diluents, buffers, flavouring agents, binders, preservatives or other ingredients that are not detrimental to the efficacy of the composition. In some embodiments, the probiotic strains may comprise from about 50% to about 90% by weight of the composition, based on the total weight of the composition including a carrier medium, or from about 60% to about 80% by weight of the composition. In particular embodiments, compositions may be gluten free and dairy free, and suitable for ingestion by vegetarians.

Compositions of the disclosure can be readily manufactured by those skilled in the art using known techniques and processes. For example, the probiotic microorganisms can be seeded from standard stock into a reactor and grown in standardized media until a predetermined cfu/g concentration is reached. The bulk material can then be drained from the reactor and dried by spray drying, lyophilization, or flatbed oven drying. The dried bacterial material can then be blended with the carrier medium and the resulting mixture can be pressed into tablets, filled into foil pouches as a granular solid, or introduced into gelatin capsules as a particulate material.

Microorganisms and the metformin or derivative or salt thereof may be suitably formulated, separately or in a single composition, for oral administration, and may be prepared according to conventional methods well known in the pharmaceutical and nutraceutical industries, such as those described in Remington's Pharmaceutical Handbook (Mack Publishing Co., NY, USA) using suitable excipients, diluents and fillers.

Compositions suitable for oral administration may be presented as discrete units (i.e. dosage forms) such as gelatine or HPMC capsules, cachets or tablets, each containing a predetermined amount of each component of the composition as a powder, granules, as a solution or a suspension in an aqueous liquid or a non-aqueous liquid, or as an oil-in-water liquid emulsion or a water-in-oil liquid emulsion.

When the composition is formulated as capsules, the components of the composition may be formulated with one or more pharmaceutically acceptable carriers such as starch, lactose, microcrystalline cellulose and/or silicon dioxide. Additional ingredients may include lubricants such as magnesium stearate and/or calcium stearate. The capsules may optionally be coated, for example, with a film coating or an enteric coating and/or may be formulated so as to provide slow or controlled release of the composition therein.

Tablets may be prepared by compression or moulding, optionally with one or more accessory ingredients. Compressed tablets may be prepared by compressing in a suitable machine the components of the composition in a free-flowing form such as a powder or granules, optionally mixed with a binder, lubricant (for example magnesium stearate or calcium stearate), inert diluent or a surface active/dispersing agent. Moulded tablets may be made by moulding a mixture of the powdered composition moistened with an inert liquid diluent, in a suitable machine. The tablets may optionally be coated, for example, with a film coating or an enteric coating and/or may be formulated so as to provide slow or controlled release of the composition therein.

The compositions may be provided to the user in a powder form. For oral administration, the composition may then be mixed with a suitable volume of an aqueous medium, typically with agitation, to dissolve the components, or produce a suspension, suitable for ingestion. Thus, the compositions may be provided to a user in a powder form, which powder may then be added by the user to any type of aqueous medium (for example water or fruit juice) and consumed there after. Alternatively, the composition may be provided as a beverage, pre-mixed with an aqueous medium such as water. In another embodiment the compositions may be added in powder form by the user to any type to a food product (for example, yoghurt) and consumed there after. In another embodiment, the compositions may simply be consumed as a powder in the absence of a drink or additional food product.

The probiotic microorganisms may be conveniently incorporated in a variety of food and/or beverage products, nutraceutical products, probiotic supplements, food additives, pharmaceuticals and over-the-counter formulations. The food or food additive may be a solid form such as a powder, or a liquid form. Specific examples of the types of beverages or foods include, but are not limited to water-based, milk-based, yoghurt-based, other dairy-based, milk-substitute based such as soy milk or oat milk, or juice-based beverages, water, soft drinks, carbonated drinks, and nutritional beverages, (including a concentrated stock solution of a beverage and a dry powder for preparation of such a beverage); baked products such as crackers, breads, muffins, rolls, bagels, biscuits, cereals, bars such as muesli bars, health food bars and the like, dressings, sauces, custards, yoghurts, puddings, pre-packaged frozen meals, soups and confectioneries.

Those skilled in the art will appreciate that single or multiple administrations of compositions disclosed herein can be carried out with dose levels and dosing regimes being determined as required depending on circumstances and requirements of the subject to be treated. The skilled addressee can readily determine suitable dosage regimes. A broad range of doses may be applicable. Dosage regimens may be adjusted to provide the optimum therapeutic response. Those skilled in the art will appreciate that the exact amounts and rates of administration of the metformin or derivative or salt thereof and of the probiotic microorganisms will depend on a number of factors such as the particular composition being administered, the age, body weight, general health, sex and dietary requirements of the subject, as well as any drugs or agents used in combination or coincidental with the compositions. For example, several divided doses may be administered hourly, daily, weekly, monthly or at other suitable time intervals or the dose may be proportionally reduced as indicated by the exigencies of the situation. Based on the teaching herein those skilled in the art will, by routine trial and experimentation, be capable of determining suitable dosage regimes on a case-by-case basis.

Compositions and methods of the present disclosure may be employed as an adjunct to other therapies or treatments for type 2 diabetes, pre-diabetes, metabolic syndrome, insulin resistance, obesity and obesity-related disorders. Accordingly compositions and methods disclosed herein may be co-administered with other agents that may facilitate a desired therapeutic outcome, for example sulfonylureas such as such as tolbutamide, glibenclamide, glipizide, chlorpropamide, or gliclazide. By “co-administered” is meant simultaneous administration in the same formulation or in two different formulations via the same or different routes or sequential administration by the same or different routes. By “sequential” administration is meant a time difference of from seconds, minutes, hours or days between the administration of the agents, compositions or treatments. Sequential administration may be in any order. Similarly, methods and compositions of the present disclosure may be employed in conjunction with lifestyle changes by the subject, such as a healthy diet and adequate exercise.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the invention.

### EXAMPLES

The following examples are illustrative of the invention and should not be construed as limiting in any way the general nature of the disclosure of the description throughout this specification.

### Example 1—Double-Blind, Randomised, Placebo-Controlled Trial

### Study Protocol

The study described herein was a pilot, single site, randomised, double blind, placebo-controlled trial. Ethical approval was granted by the Sydney Local Health District Human Research Ethics Committee, RPA Hospital, Sydney, Australia. The study was carried out according to the Declaration of Helsinki, the National Health and Medical Research Council National Statement on Ethical Conduct in Research Involving Humans and the Notes for Guidance on Good Clinical Practice as adopted by the Australian Therapeutic Goods Administration and the International Conference on Harmonisation Good Clinical Practise guidelines. Written informed consent was obtained from participants before enrolment.

Individuals were eligible for the study if the following criteria were met: (i) aged ≥18 years; (ii) BMI≥25 kg/m²; (iii) pre-diabetes or type 2 diabetes mellitus diagnosed within the previous 12 months (criteria for the diagnosis of pre-diabetes and type 2 diabetes were based on the guidelines of the American Diabetes Association); (iv) treated by diet alone or diet plus metformin and; (v) willingness to adhere to the study protocol (no yogurt, fermented food, dietary supplements, probiotics or prebiotics) for the duration of the study.

Individuals were excluded from the study if any of the following factors applied: (i) type 1 diabetes mellitus; (ii) type 2 diabetes mellitus diagnosed for more than 12 months; (iii) taking anti-obesity drugs or blood glucose-lowering medications (i.e. sulfonylureas, alpha-glucosidase inhibitors, thiazolidinediones and glucagon-like peptide-1 analogues) other than metformin; (iv) presence of concomitant gastrointestinal disorders (irritable bowel syndrome, inflammatory bowel disease and coeliac disease); (v) recent use (within the previous four weeks) of antibiotics and dietary supplements (fish oil, probiotics, prebiotics, multivitamins, minerals, nutraceuticals and herbal preparations); (vi) pregnancy, breast feeding or planning to become becoming pregnant; (vii) alcohol abuse or the use of any illicit drugs and; (viii) clinical evidence of active infection or any severe illness unrelated to diabetes.

The active product administered was a capsule containing 5×10¹⁰ colony forming units (cfu) of a multi-strain probiotic combination of Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii. The placebo was a capsule containing 50 mg maltodextrin. The probiotic and placebo capsules were opaque white in colour and looked and smelled identical.

Participants were randomised to the probiotic (intervention) or placebo group without stratification using computer-generated random numbers (FileMaker Pro). Participants and study investigators were blinded to treatment allocation. Blinding remained intact until the statistical testing of the outcomes.

Enrolled participants in each group were required to: take two capsules twice per day (20 minutes before breakfast and dinner) with cold non-carbonated water (not to be mixed or taken with hot drinks or foods (as heat and stomach acids can reduce the stability of the probiotic bacteria) for the 12 week course of the study; store the capsules in the refrigerator at 4-6° C.; record the number of capsules taken each day; bring all capsules remaining in the bottle to the subsequent visit and; avoid eating/drinking yoghurt, fermented food, dietary supplements (i.e. vitamins, minerals, nutraceuticals, herbal preparations, probiotics, prebiotics or fish oils) and antibiotics (unless recommended by a health professional). Capsule counting, at weeks six and 12, was used to assess the participant's compliance. A participant was deemed to be non-compliant if they took less than 80% of the study product on both occasions.

Changes were assessed in the following biomarkers and measurements from baseline (week 0 at initiation of the study) to 12 weeks: (i) plasma HbA1c, 2-h post-load glucose, triglycerides, free fatty acids, total cholesterol, HDL-c, LDL-c, high-sensitive CRP, LPS and zonulin; (ii) insulin resistance measured by the homeostatic model assessment for insulin resistance (HOMA-IR) and insulin sensitivity index of Matsuda (ISI-M); (iii) faecal microbial profile and short chain fatty acids; (iv) weight, BMI, waist circumference and waist and hip ratio; (v) gastrointestinal symptoms and; (vi) blood pressure. Differences in all biomarkers/measurements were determined between participants with pre-diabetes and participants with type 2 diabetes in the intervention (probiotic) group and the placebo group from baseline to 12 weeks. Differences were determined between participants taking and not taking metformin in the intervention (probiotic) group and the placebo group from baseline to 12 weeks on glycaemic parameters, insulin resistance, LPS, zonulin, faecal microbial profile, short chain fatty acid levels and gastrointestinal symptoms.

### Sampling and Analysis

Blood samples were collected after fasting overnight at baseline and 12 weeks. Participants who did not have type 2 diabetes underwent a 75-g oral glucose tolerance test (OGTT) to assess glucose and insulin levels at three time-points (0 min, 60 min and 120 min). Insulin resistance, calculated by ISI-M and HOMA-IR, was computed using the glucose and insulin results from the OGTT and fasting test, respectively. Blood samples were analysed by Douglass Hanly Moir Pathology for glucose, insulin, HbA1c, lipids and hs-CRP levels.

Stool (faecal) samples were collected at baseline and 12 weeks using a stool specimen collection kit. This collection kit included an instruction booklet for the stool sample collection and transportation, ice packs, gloves, a sterile container, sealed plastic pouch, cool box and an AnaeroGen™ Compact sachet that preserved the microbiological characteristics of the sample for 72 hours. Participants brought the stool sample to the clinic and a one-gram sample was stored at −80° C. for faecal microbial and short chain fatty acid analysis.

Faecal bacterial profiles were determined using the Ion S5 next-generation sequencing platform (Thermo Fisher) and faecal yeasts were identified by matrix-assisted laser desorption ionization time-of-flight mass spectrometry (MALDI-TOF). The MALDI-TOF analysis was performed 24-48 hours after sample collection by a commercial laboratory (Bioscreen). To assess intestinal bacteria, faecal DNA was extracted seven days after sample collection using the QIAamp DNA stool kit (Qiagen) according to manufacturer's instructions. After extraction, DNA was quantified in a Qubit 2.0 Fluorometer (Invitrogen, Thermo Fisher). Hypervariable 16S rRNA gene regions were targeted using the Ion 16S Metagenomics Kit (Thermo Fisher). Two sets of primer pools were used to amplify seven of nine hypervariable regions of the 16S rRNA gene (Pool 1: V2-4-8; Pool 2: V3-6, 7-9) by PCR using Environmental Master Mix v2.0. PCR products were quantified and their quality was evaluated by electrophoretic fractionation in 2% agarose gels (E-Gel, EX Agarose Gel, Thermo Fisher). 100 ng of PCR amplification products were used to create a library using an enzymatic-based kit (Ion Plus Fragment Library Kit, Thermo Fisher). Template preparation was performed using the Ion Chef™ System according to the manufacturer's instructions. During this process, library fragments were clonally amplified onto ion sphere particles/beads by emulsion PCR. Next-generation sequencing of 16S rRNA gene amplicons was conducted on the Ion S5 system (Thermo Fisher) using a 520 chip. Eight barcoded samples were processed on each chip. Ion Reporter™ software was used for data analysis.

Statistical analyses were conducted using intention-to-treat and missing data was imputed with baseline values for a conservative estimate (i.e. no change). All data was checked for normal distribution using the Shapiro-Wilk test. Descriptive statistics were presented as mean±standard deviation or median with interquartile range (IQR), as appropriate. HbA1c and insulin resistance were analysed using generalised linear models and the covariates of age, sex and baseline values were added to the model. Other outcomes were analysed using independent Student's t test or the Mann-Whitney test to compare the study groups at week 12. Paired Student's t test or Wilcoxon matched-pairs signed-rank was used to analyse differences between baseline and endpoint values. Three subgroup analyses were completed between those participants taking metformin, participants classified as having pre-diabetes or type 2 diabetes, and those participants defined as compliant. Statistical analyses were performed using SPSS Statistics version 22 (IBM).

For intestinal microbiome (microbiota) analysis, sequenced data was interpreted using the bioinformatics tools programmed in Ion Reporter™ software. Quantitative Insights Into Microbial Ecology (QIIME) algorithms determined the bacterial diversity within samples (alpha diversity) and between samples (beta diversity). For alpha diversity, the Chaol index and operational taxonomic units (OTU) tables were used to assess bacteria richness and relative abundance, respectively. For beta diversity, Adonis test (a more robust version of the known permutational analysis of variance, PERMANOVA) and principal component analysis (PCoA) at the family, genus and species level were performed to examine differences in the microbial composition between the two groups. Correlations between a participant's anthropometric measurements and metabolic markers with the abundance of microbial species was conducted using DESeq2 software. Briefly, DESeq2 tests for differential abundance by use of negative binomial generalized linear models obtaining maximum likelihood estimates for an OUT's log 2-fold changes between two or more conditions. Moreover, a linear model including variations induced by condition (pre-diabetes or type 2 diabetes), group (probiotic or placebo) and time-point (baseline or week-12) was employed. A P value of less than 0.05 was considered significant.

### Baseline Characteristics

Participants were on average 59 years old, their mean BMI was above 30 kg/m², 97% of the participants had central obesity, 68% had insulin resistance (HOMA-IR≥2.5), 40% had plasma total cholesterol concentrations >5.2 mmol/L, 58% had plasma LDL-cholesterol >2.6 mmol/L, 50% of women and 68% of men had low plasma HDL-cholesterol (<1.4 mmol/L and <1.15 mmol/L, respectively), 20% had plasma triglyceride concentrations >2.3 mmol/L, 34% had plasma free fatty acids >720 μmol/L, 50% had systolic blood pressure >130 mmHg, 30% had diastolic blood pressure >80 mmHg, 25% had elevated plasma hs-CRP >5 mg/L. Moreover, 68% had plasma LPS above values considered normal in adults (0.15-0.35 EU/ml) and 34% had plasma zonulin concentrations above the normal range (30-200 mg/mL). A total of 20 participants (10 in each group) were classified as non-compliant as they took less than 80% of the study product. Therefore, sub-group analysis was performed on 40 compliant participants (20 in each group).

TABLE 1
Baseline characteristics of participants by group and condition/disease

| Placebo | | | Probiotic | | |
|---|---|---|---|---|---|
| | Pre- | | | Pre- | |
| All | diabetes | T2DM | All | diabetes | T2DM |
| (n = 30) | (n = 19) | (n = 11) | (n = 30) | (n = 17) | (n = 13) |

Gender

| | | | | | | |
|---|---|---|---|---|---|---|
| Female | 19 (63%) | 15 (79%) | 4 (36%) | 13 (43%) | 7 (41%) | 6 (46%) |
| Male | 11 (37%) | 4 (21%) | 7 (64%) | 17 (57%) | 10 (59%) | 7 (54%) |
| Age (years) | 56.1 ± 12.3 | 56.5 ± 11.1 | 55.5 ± 14.7 | 61.4 ± 8.9 | 62.9 ± 9.3 | 59.3 ± 8.3 |

Medications

| | | | | | | |
|---|---|---|---|---|---|---|
| Metformin | 14 (47%) | 7 (37%) | 7 (64%) | 14 (47%) | 4 (24%) | 10 (77%) |
| Lipid- | 8 (27%) | 5 (26%) | 3 (27%) | 15 (50%) | 10 (59%) | 5 (38%) |
| lowering | | | | | | |
| BP- | 14 (47%) | 9 (47%) | 5 (45%) | 13 (43%) | 7 (41%) | 6 (46%) |
| lowering^{a} | | | | | | |

Body composition

| | | | | | | |
|---|---|---|---|---|---|---|
| Height (m) | 1.7 ± 0.1 | 1.6 ± 0.1 | 1.7 ± 0.1 | 1.7 ± 0.1 | 1.7 ± 0.1 | 1.7 ± 0.1 |
| Body weight | 101.7 ± 21.9 | 97.5 ± 19.6 | 108.9 ± 24.9 | 100.1 ± 20.4 | 97.3 ± 14 | 103.8 ± 26.8 |
| BMI (kg/m²) | 36.3 ± 7.5 | 35.9 ± 5.7 | 37.2 ± 10.0 | 35.5 ± 6.2 | 33.9 ± 3.9 | 37.6 ± 8.0 |

Glycaemia

| | | | | | | |
|---|---|---|---|---|---|---|
| FPG | 6.3 ± 1.6 | 5.6 ± 0.6 | 7.4 ± 2.0 | 7.0 ± 3.0 | 5.9 ± 0.6 | 8.6 ± 4.1 |
| (mmol/L) | | | | | | |
| HbA1c (%) | 6.3 ± 1.1 | 5.8 ± 0.4 | 7.0 ± 1.4 | 6.6 ± 1.4 | 5.8 ± 0.3 | 7.6 ± 1.6 |

Lipids

| | | | | | | |
|---|---|---|---|---|---|---|
| Total | 5.4 ± 1.3 | 5.2 ± 1.2 | 5.6 ± 1.2 | 4.9 ± 1.1 | 5.0 ± 0.3 | 4.8 ± 0.9 |
| cholesterol | | | | | | |
| (mmol/L) | | | | | | |
| Triglycerides | 1.9 ± 1.2 | 1.8 ± 1.1 | 2.1 ± 1.3 | 1.8 ± 0.9 | 1.6 ± 0.6 | 2.1 ± 1.1 |
| (mmol/L) | | | | | | |

Blood pressure

| | | | | | | |
|---|---|---|---|---|---|---|
| Systolic | 127.8 ± 12.5 | 126.4 ± 14 | 130 ± 9.3 | 133.0 ± 10.8 | 133.7 ± 7.9 | 132.1 ± 14.1 |
| (mmHg) | | | | | | |
| Diastolic | 81.6 ± 6.3 | 80.4 ± 6.4 | 83.6 ± 5.9 | 79.8 ± 7.3 | 79.6 ± 7.8 | 79.9 ± 6.8 |
| (mmHg) | | | | | | |

Inflammation/intestinal permeability

| | | | | | | |
|---|---|---|---|---|---|---|
| hs-CRP | 2.7 (5.2) | 2.6 (5.5) | 3.0 (5.3) | 3.0 (3.4) | 2.5 (2.2) | 4.7 ± 11.7 |
| (mg/L) | | | | | | |
| LPS | 0.6 ± 0.3 | 0.5 ± 0.4 | 0.6 ± 0.3 | 0.5 ± 0.2 | 0.5 ± 0.2 | 0.5 ± 0.3 |
| (EU/mL) | | | | | | |
| Zonulin | 207.8 ± 172.9 | 217.2 ± 183.9 | 191.5 ± 159.3 | 203.6 ± 171.9 | 171.3 ± 106.2 | 249.2 ± 234.6 |
| (mg/dL) | | | | | | |

Results are mean ± SD or median (IQR) or number and percentage where appropriate.
FPG: fasting plasma glucose;
LPS: lipopolysaccharide;
^{a}blood pressure lowering medication: ACE inhibitors, angiotensin II receptor blockers, beta-blockers and calcium channel blockers

### Outcomes

Examples 2 and 3 below detail outcomes determined from the present study Results of the present study are detailed in Examples 2 and 3 below.

### Example 2—Effect of Metformin and a Multi-Strain Probiotic Combination on Intestinal Microbiota in Type 2 Diabetic and Pre-Diabetic Subjects

The baseline intestinal microbial profile of participants, as determined by 16S rRNA genes of microbes in faecal samples showed that 99% of the sequences were distributed among three bacterial phyla the Bacteroidetes, Firmicutes and Proteobacteria. The remaining 1% was composed primarily of Actinobacteria, Verrucomicrobia, Fusobacteria and Tenericutes.

Operational taxonomic unit (OTU) tables were extracted from Ion Reporter software and the data from MALDI-TOF reports analysed to assess if the abundance of specific microbial species changed after the probiotic intervention. A significant increase was found in the probiotic group compared to the placebo group (p<0.05) in the abundance of beneficial microorganisms including Lactobacillus gasseri, Bifidobacterium longum, Bifidobacterium bifidum and Saccharomyces boulardii, in addition to a decrease in the butyrate-producing bacteria Roseburia intestinalis and Roseburia inulinivorans and in the gram-positive bacteria Eubacterium ramulus and Ruminococcus gauvreauii. A sub-analysis in compliant participants showed that, in addition to the above changes, an increase of Bifidobacterium breve and Faecalibacterium prausnitzii abundance occurred in the probiotic group and not in the placebo group (p<0.05).

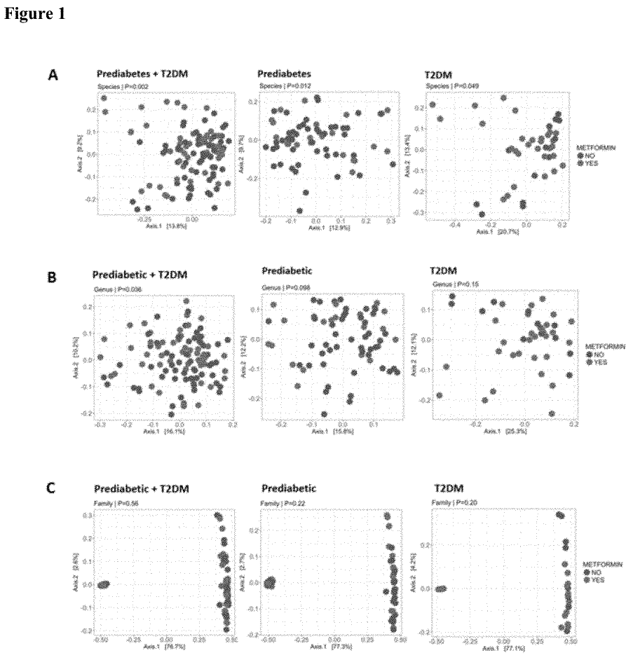

Beta diversity analysis showed that the intestinal microbial community in participants taking metformin differed from those not taking metformin, with the difference being significant only at a species and genera level (p=0.002 and 0.036, respectively; FIGS. 1A and 1B). At the family level, metformin had no effect on intestinal microbiota (p=0.56; FIG. 1C).

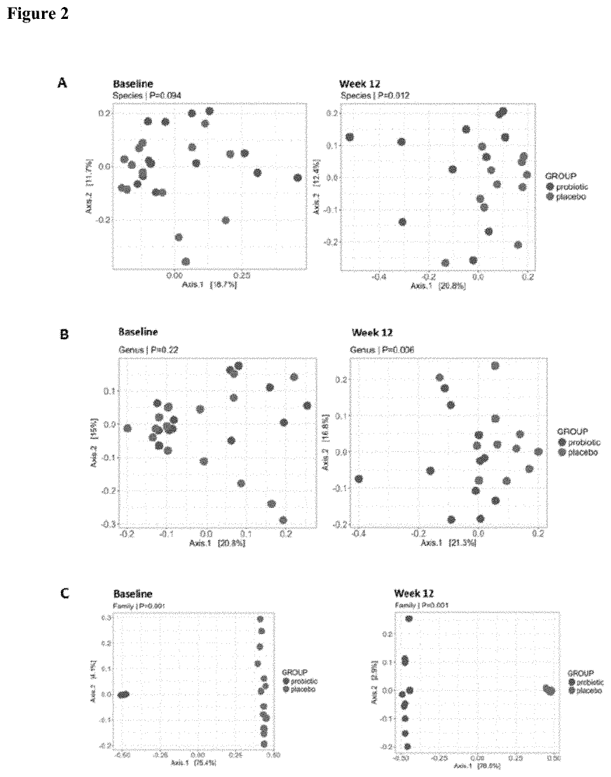

In participants taking metformin, probiotic supplementation had a significant effect on the intestinal microbial community compared to those on placebo at the species, genera and family level (p=0.012, 0.006 and 0.001, respectively; FIG. 2). This suggests the probiotic and metformin have a combined effect on the microbiome. Differences in the relative abundance of Roseburia, Phascolarctobacterium and Bifidobacterium genera were observed between the probiotic and placebo groups. A higher abundance of Bifidobacterium and Phascolarctobacterium and a lower abundance of Roseburia were found in participants taking metformin in the probiotic group compared to those in the placebo group (p<0.05). At the species level a lower relative abundance of Akkermansia municiphila was observed in those taking metformin in the placebo group in contrast to the probiotic group.

### Example 3—Effects of a Multi-Strain Probiotic Combination on Glycaemic Parameters in Type 2 Diabetic and Pre-Diabetic Subjects

Subgroup analyses in participants diagnosed with type 2 diabetes showed reductions in HOMA-IR associated with the probiotic intake after adjustment for baseline HOMA-IR, metformin, sex and age (regression coefficient −2.64; 95% CI: −4.58, −0.70; p=0.01). Within group comparisons also revealed that HbA1c levels and insulin sensitivity indices improved in the probiotic group, while in the placebo group no significant changes were found. Changes within the probiotic group were also found in subgroup analyses. In participants with type 2 diabetes in the probiotic group, the mean change of HbA1c and HOMA-IR was −0.7±0.8% (p=0.025) and −2±2.7 (p=0.05), respectively. In contrast, no significant changes were found in participants with type 2 diabetes in the placebo group (HbA1c: −0.4±0.8, p=0.14 and +0.7±2.4, p=0.3).

1. A method for modulating the intestinal microbial profile in a type 2 diabetic or pre-diabetic subject, the method comprising administering to the subject metformin or a derivative or salt thereof and one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii, whereby the modulation of the intestinal microbial profile resulting from said administration comprises a decrease in abundance of pathogenic bacteria or bacteria that do not tolerate the host and/or an increase in the abundance of bacteria that tolerate the host.

2. A method according to claim 1, wherein the intestinal microbial profile is determined by sequencing of faecal DNA from one or more faecal samples obtained the subject.

3. A method according to claim 1 or 2, wherein modulation of the intestinal microbial profile comprises an increase in abundance of one or more Bifidobacterium spp., Phascoarctobacterium spp. and/or Akkermansia spp.

4. A method according to any one of claims 1 to 3, wherein modulation of the intestinal microbial profile comprises a decrease in abundance of one or more Roseburia spp.

5. A method for reducing intestinal dysbiosis in a type 2 diabetic or pre-diabetic subject, the method comprising administering to the subject metformin or a derivative or salt thereof and one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

6. A method according to any one of claims 1 to 5, wherein the metformin or derivative or salt thereof is administered in conjunction with a multi-strain probiotic combination comprising Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

7. A method according to any one of claims 1 to 6, wherein the metformin or derivative or salt thereof and the one or more probiotic microorganisms or the multi-strain probiotic combination are administered simultaneously or sequentially.

8. A method according to any one of claims 1 to 6, wherein the metformin or derivative or salt thereof and the one or more probiotic microorganisms or the multi-strain probiotic combination are formulated in the same composition.

9. A method for treating type 2 diabetes or a metabolic complication or condition associated therewith in a subject, the method comprising administering to the subject one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

10. A method according to claim 9, wherein treating the type 2 diabetes or metabolic complication or condition associated therewith comprises decreasing glycosylated haemoglobin (HbA1c) levels, reducing BMI, improving insulin sensitivity, or reducing insulin resistance.

11. A method for delaying or inhibiting the onset of type 2 diabetes in a pre-diabetic subject, the method comprising administering to the subject one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

12. A method according to claim 11, wherein delaying or inhibiting the onset of type 2 diabetes comprises decreasing glycosylated haemoglobin (HbA1c) levels, reducing BMI, improving insulin sensitivity, or reducing insulin resistance.

13. A method according to any one of claims 9 to 12, wherein the subject is administered a multi-strain probiotic combination comprising Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.

14. A method according to any one of claims 9 to 13, wherein the subject is also administered metformin or a derivative or salt thereof.

15. A composition for treating type 2 diabetes or a metabolic complication or condition associated therewith, for delaying or inhibiting the onset of type 2 diabetes, or for modulating the intestinal microbiota in a type 2 diabetic or pre-diabetic subject, the composition comprising metformin or a derivative or salt thereof and one or more probiotic microorganisms selected from Lactobacillus plantarum, Lactobacillus bulgaricus, Lactobacillus gasseri, Bifidobacterum breve, Bifidobacterium animalis subsp. lactis, Bifidobacterium bifidum, Streptococcus thermophilus and Saccharomyces boulardii.
